(19) United States
(12) Patent Application Publication
(10) Pub. No.: US 2005/0036979 A1
(43) Pub. Date: Feb. 17, 2005

(54) Fibrinogen adsorber III

(76) Inventors: Wolfgang Metzger, Bous (DE); Veit Otto, St. Wendel (DE); Walter Ruger, Schenklengsfeld (DE); Martin Schimmel, Wollstadt (DE)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/859,923

(22) Filed: Jun. 3, 2004

(30) Foreign Application Priority Data
Jun. 4, 2003 (DE) 103 25 304.1-43

(51) Int. Cl. A61K 31/74; C08L 33/02

(52) U.S. Cl. 424/78.17; 525/221

(57) ABSTRACT

This invention relates to an adsorbent for lowering the concentration of fibrinogen and/or fibrin in the blood or blood plasma, encompassing an organic matrix with synthetic side chains covalently bound to the matrix and exhibiting terminal vicinal hydroxy groups formed by the hydrolysis of terminal epoxy groups, which synthetic side chains are free of peptides and of any groups of aromatics. The invention further relates to a method for preparing the adsorbent, and to the use of the adsorbent for producing an adsorber serving to lower the concentration of fibrinogen and/or fibrin in the blood or blood plasma.

This invention relates to an adsorbent for lowering the concentration of fibrinogen and/or fibrin in the blood or blood plasma, encompassing an organic matrix with synthetic side chains covalently bound to the matrix and exhibiting terminal vicinal hydroxy groups formed by the hydrolysis of epoxy groups, said synthetic side chains being free of peptides and devoid of any aromatic groups. The invention further relates to a method for preparing the adsorbent and to the use of the adsorbent for producing an adsorber serving to lower the concentration of fibrinogen and/or fibrin in the blood or blood plasma.

Adsorbents are widely used in medical technology. Many publications discuss adsorbers containing adsorbents that remove low-density lipoproteins (LDL) from the blood, or lower their concentration, as described in DE 39 32 971. The latter refers to the adsorber material as an organic carrier with a fixed particle size and exclusion limit and carrying on its surface a ligand to which the LDL molecule is bonded.

DE 197 29 591 describes the use of a ligand for fibrinogen and/or fibrin, aimed at curing or preventing illnesses attributable to an excessive fibrinogen concentration in the blood. DE 197 29 591 defines the ligand as a substance that specifically attaches to fibrinogen and/or fibrin and is preferably a peptide with three to 10 amino acids.

Artificial Organs, vol. 20, No. 9 (1996), pages 986-990, discusses the reduction of concentrations of plasma fibrinogen, immunoglobulin G (IgG) and immunoglobulin M (IgM) through immunoadsorption therapy using tryptophan or phenyl alanine adsorbents.

Immunoadsorption therapy employs adsorption columns whose carriers are in the form of spherical polyvinyl alcohol (PVA) gel particles. On their surface these PVA gel particles carry either tryptophan or phenyl alanine as the amino acid ligand which, by way of spacers, is covalently bound to the PVA. The plasma separated from blood cells is channeled through the adsorption column and is then reunited with the blood cells prior to being reintroduced in the patient. This type of immunoadsorption therapy simultaneously and significantly reduces the concentrations of fibrinogen, IgG and IgM.

As much as adsorption has by now become a routine clinical tool for alleviating illnesses, it must meet ever more demanding selectivity requirements. What this means is that, for one, the adsorbers must not adsorb any, or as few as possible, of the proteins the human body needs while at the same time reducing the concentration of harmful proteins to a point that optimizes the effectiveness of the extracorporal treatment to which the patient is subjected.

It has been known for some time that a number of illnesses are attributable to a lack of microcirculation of the blood. Examples of such illnesses are given below.

CNS/central nervous system: Stroke, TIA (transient ischemic attack), PRIND (prolonged reversible ischemic neurological deficit), chronic vascular disorders of the CNS, chronic intracranial perfusion disorders, chronic extracranial perfusion disorders, cerebrovascular perfusion disorders, dementia, Alzheimer's disease, severe central vertigo

Eyes: Chronic perfusion disorder, acute vascular occlusion

Ears: Apoplectiform deafness, inner-ear-related vertigo, Meniere's disease

Lungs: Primary pulmonary hypertension, veno-occlusive lung diseases, thrombotic primary pulmonary hypertension, thromboembolic diseases of the large vessels

Heart: Transplant vasculopathy, acute myocardial infarction, unstable angina pectoris, small vessel disease of the heart, inoperable severe coronary heart disease, myocardiopathy

Abdomen: Ortner's disease

Kidneys: Renal vasculopathy, glomerulonephritis, chronic renal insufficiency

Peripheral arterial occlusive diseases

Acute vascular occlusions

Vasculitis

Septic shock

Disseminated intravascular coagulation (DIC) by other causes such as oncogenesis Diabetes type I and II

Diabetic retinopathy

Diabetic neuropathy

Diabetic nephropathy

To date, these illnesses have been treated primarily with medication, often curing the symptoms only. The approaches so far taken in an effort to treat and improve the microcirculation and the rheology of the blood have consisted in a plasma exchange, a heparin-induced extracorporal LDL-cholesterol precipitation (HELP) and in the adsorption of fibrinogen with the aid of a ligand to which the fibrin and/or fibrinogen is specifically bonded. DE 197 29 591 describes the use of a ligand of that type. The ligands mentioned are peptides preferably including 3 to 10 amino acids, with the sequence of particular preference said to be glycine-proline-arginine-proline-X.

The synthetic production of peptides, however, is an awkward and costly undertaking, making their use as the ligand of a particular adsorber quite expensive. Moreover, above a certain length, peptides begin to trigger antibody reactions, so that over the long term repeated application can lead to intense immune reactions. To be sure, the shortest possible peptide oligomers are being used in order to minimize the immune defense, but immunogeneity can never be totally prevented. Then, too, leakage i.e. an unnoticed separation of peptide pieces is particularly dangerous, given that peptides as a component of the body's intrinsic structures constitute bioactive molecules.

Immunoadsorption therapy on its part, as described in Artificial Organs, vol. 20, no. 9 (1996), pages 986-990, employs tryptophan or phenyl alanine as the amino acids to be attached to the PVA gel particles, making it equally complex and costly.

Moreover, in the process of that therapy, substances that should not be removed from the plasma, such as IgG and IgM, are separated from the plasma in an amount comparable to that of fibrinogen.

EP-A1-1 132 128 and EP-A1-1 132 129 describe peptide-free adsorber beads. It has been found, however, that for practical applications the adsorber beads described in EP-A1-1 132 128 and EP-A1-1 132 129 exhibit too strong a bond with thrombocytes. It follows that, before that type of adsorber beads can be used for fibrinogen reduction in whole blood or blood components as well as PRP (platelet-rich plasma), some improvements are needed especially in terms of a diminished affinity to thrombocytes, in parallel with a strong affinity to fibrinogen.

It is therefore the objective of this invention to introduce an adsorbent for lowering the concentration of fibrinogen and/or fibrin in the blood or blood plasma, that offers a high elimination rate, is easy to produce and is biocompatible, does not provoke an immune defense and, compared to prior-art adsorbents, is designed to exhibit less affinity to thrombocytes while at the same time offering high affinity to fibrinogen.

This objective is achieved with the forms of implementation specified in the claims.

Specifically, this invention relates to an adsorbent for lowering the concentration of fibrinogen and/or fibrin in blood or blood plasma, encompassing an organic matrix with synthetic side chains that are covalently bound to the matrix and exhibit terminal vicinal hydroxy groups formed by the hydrolysis of terminal epoxy groups, where the said synthetic side chains are free of peptides and do not contain any aromatic groups.

Surprisingly, it has been revealed that an adsorbent encompassing an organic matrix with synthetic side chains that are covalently bound to the matrix and exhibit terminal vicinal hydroxy groups formed by the hydrolysis of terminal epoxy groups, brings about a substantial reduction of the fibrinogen level, improving post-treatment microcirculation, and that, compared to prior-art adsorbents, a lower affinity to thrombocytes is obtained at the same time.

An important aspect for the use of this type of adsorbent is its ability to be sterilized, and in particular to be thermally sterilizable, since the treated blood is to be returned to the patient without posing the risk of causing sepsis or infections. In contrast to the matrix according to this invention, it being an organic matrix with stable side chains, the peptides and amino acids employed in prior art are not thermally or chemically stable. The synthetic side chains covalently bound to the matrix are therefore completely free of peptides.

It has also been found that the presence of aromatic groups in the side chains unfavorably affects the bonding capacity while additionally diminishing the selectivity of the adsorbent with regard to fibrinogen and/or fibrin. Accordingly, the covalently bound synthetic side chains of the adsorbent according to this invention contain no aromatic groups.

The term “synthetic” used in this context signifies that for introducing the side chains in the matrix no biological material is used, and especially no peptides, i.e. no dipeptides, tripeptides, oligopeptides, polypeptides, or proteins (macropeptides) even if these were synthetically produced. As an example, the synthetic side chain covalently bound to the matrix can have the following structure: 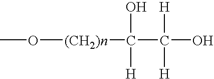
where n is an integer in the range from 1 to 18, preferably 1 to 10, and most desirably 1.

In principle there are several materials that can serve as the organic matrix, for instance carbohydrates or such organic matrices as acrylate or methacrylate copolymers as well as polyamides. Within the scope of this invention the preferred organic matrix is a copolymer derived from (meth)acrylic acid esters. The term “(meth)acrylic” is intended to cover the corresponding acrylic as well as methacrylic compounds. More preference for the organic matrix is given to a copolymer derived from at least one epoxy(meth)acrylate and at least one cross-linking agent, selected from the group consisting of alkylene di(meth)acrylates and polyglycol di(meth)acrylates. Copolymers of that type are preferably produced by suspension polymerization.

The matrix most preferred is a random copolymer produced by the polymerization of the monomeric units
(i) glycidyl methacrylate in an amount of 5 to 95% by weight, preferably 40 to 80% by weight, and most desirably 60% by weight, and
(ii) ethylene glycol dimethacrylate in an amount from 5 to 95% by weight, preferably 20 to 60% by weight and most desirably 40% by weight,
   relative in each case to the total weight of the monomeric units.

As part of this invention, the organic matrix containing epoxy groups (oxirane groups), as for instance the copolymer referred to above, is hydrolyzed in a manner, according to the invention, as to form terminal vicinal hydroxy groups, meaning 1,2-diol groups. The hydrolysis may be performed for instance by incubation, at a temperature in the range from around room temperature to 90° C. for a duration ranging from about 30 minutes to 24 hours, in 1 to 8 M NaOH but preferably 4 M NaOH. The hydroxyl number of the organic matrix is preferably in the range from 50 to 1000 μmol/g relative to the dry weight of the adsorber material. The hydroxyl number can be controlled as a function of the epoxy-group content of the copolymer employed.

The matrix may be in the form of spherical non-aggregated particles, so-called beads, or of fibers or of a membrane, with a porous nature of the matrix increasing its surface area. Porosity can be obtained for instance by admixing pore-forming substances such as cyclohexanol or 1-dodecanol to the reaction mixture of the suspension polymerization. It will also be advantageous for the matrix to have an exclusion limit of at least 10⁷ Daltons, allowing the fibrinogen to penetrate into the pores together with the plasma and to reach the side chains of the organic matrix that contain the terminal vicinal hydroxy groups.

In another embodiment of the invention, the adsorber according to the present invention is used in whole blood based on an appropriate selection of the carrier matrix. To that effect, the matrix consists of non-aggregated spherical particles whose particle-size distribution is preferably in the range from 50 to 250 μm and its pore-radius distribution is in the range from 10 to 200 nm. That allows blood cells to make contact with the adsorber material without clogging the column and without an unacceptable volume of cells being held back or caused to agglomerate. The adsorbent according to this invention makes that possible by virtue of the size and spherical shape of the beads, in that the cells slide along the smooth outer surface of the beads, minimizing thrombocyte adhesion while still allowing the plasma with the fibrinogen to penetrate into the pores.

This obviates the need for extracorporal procedures such as the separation of blood cells, the processing of the isolated plasma and the recombination of the blood components, thus enhancing the biocompatibility of this method whereby, for one example, the threat of a complement activation is further reduced to a significant extent. Doing away with extracorporal procedures shortens treatment times and simplifies the method, which in turn makes for improved safety and patient comfort.

An adsorber employing the adsorbent according to this invention encompasses a casing preferably in the form of a tube or column that is filled with the adsorbent. In view of the typical throughput amounts of blood or blood plasma on the one hand and of the effectiveness of the adsorber according to the present invention on the other, the adsorber is preferably designed for a capacity of 250 to 1250 ml. The adsorber permits single-, twin- or multi-unit operation. Using two or more adsorbers allows for alternating operation whereby one adsorber is filled with blood or blood plasma while the other adsorber is regenerated, thus further enhancing the efficient use of the adsorber according to the invention. The adsorber is preferably designed with a casing featuring at its top end an inlet through which the blood or blood plasma is fed to the adsorbent, in which case the outlet is situated on the bottom of the adsorber casing.

To prevent undesirable substances such as those originating in the adsorbent material from being recirculated into the patient's blood stream together with the treated blood or blood plasma, it is desirable to provide the outlet in the adsorber casing with a filter, preferably a particle filter.

As another object, this invention also relates to a method for producing the above-described adsorbent for lowering the concentration of fibrinogen and/or fibrin in the blood or blood plasma, said method comprising the following steps:
(a) Preparation of the organic matrix with synthetic side chains covalently bound to the matrix and exhibiting terminal epoxy groups;
(b) Hydrolysis of the epoxy groups of the organic matrix with the concurrent introduction of terminal vicinal hydroxy groups into the synthetic side chains covalently bound to the matrix; and
(c) where appropriate, heat treatment of the material obtained in step (b) at a temperature of ≧100° C.

By applying a method along this concept an adsorbent material can be obtained that is easy to produce, is biocompatible and does not trigger any immune defense. Surprisingly, the simple hydrolysis of the organic matrix with synthetic side chains covalently bound to the matrix and exhibiting terminal epoxy groups as described above, results in an adsorber material that displays an excellent fibrinogen bonding capacity with a concurrently diminished affinity to thrombocytes.

An important aspect for the use of this type of adsorbent is its ability to be sterilized, and in particular to be thermally sterilizable at a temperature in the range preferably from 100 to 140° C. or, more desirably, at 121° C., for a duration for instance of 20 to 60 minutes, since the treated blood is to be returned to the patient without posing the risk of causing sepsis or infections. As a particular advantage, heat treatment and sterilization can be performed in a single procedural step. Moreover, the adsorbent produced in accordance with this invention has been found to be biocompatible.

The matrix employed in applying the method per this invention preferably contains epoxy groups in an amount of 25 to 500 μmol/g as related to the dry weight of the adsorber material. The organic matrix is preferably a copolymer derived from at least one epoxy(meth)acrylate and at least one cross-linking agent, selected from the group comprising alkylene di(meth)acrylates and polyglycol di(meth)acrylates. This type of copolymer can be produced especially by a suspension polymerization as described for instance in WO 95/26988.

The matrix most preferred is a random copolymer produced by the polymerization of the monomeric units
(i) glycidyl methacrylate in an amount of 5 to 95% by weight, preferably 40 to 80% by weight, and most desirably 60% by weight, and
(ii) ethylene glycol dimethacrylate in an amount from 5 to 95% by weight, preferably 20 to 60% by weight and most desirably 40% by weight,
   relative in each case to the total weight of the monomeric units.

The hydrolysis as part of the method according to this invention may be performed for instance by incubation, at a temperature in the range from around room temperature to 90° C. for a duration ranging from about 30 minutes to 24 hours, in 1 to 8 M NaOH but preferably 4 M NaOH.

The following will explain this invention in more detail by way of an example without being limited to the latter.

### EXAMPLE

A copolymer was produced from ethylene glycoldimethacrylate (EGDMA) and glycidyl methacrylate (GMA) by suspension polymerization using the method described in WO 95/26988. A mixture of 181 g EGDMA and 272 g GMA and the solvents cyclohexanol (542 g) and dodecanol (54 g) together with the initiator AIBN (1% by weight as related to the total weight of the monomeric units) was stirred into 3075 ml of a polyvinyl alcohol solution in water. The resulting mixture was polymerized for 2 hours at 54° C. After the polymerization of the residual monomers at 75° C. and 88° C. the material thus obtained was washed in isopropanol and water and fractionated.

The product was then hydrolyzed with 4 M NaOH at a temperature of about 70° C. for a duration of 30 minutes.

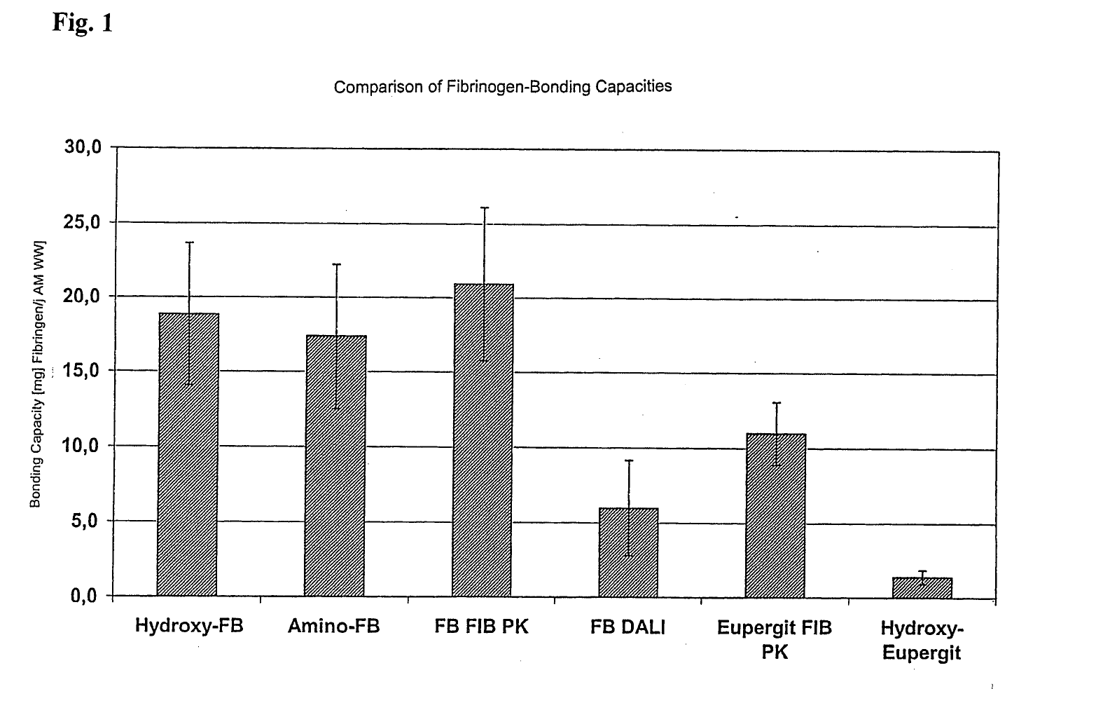

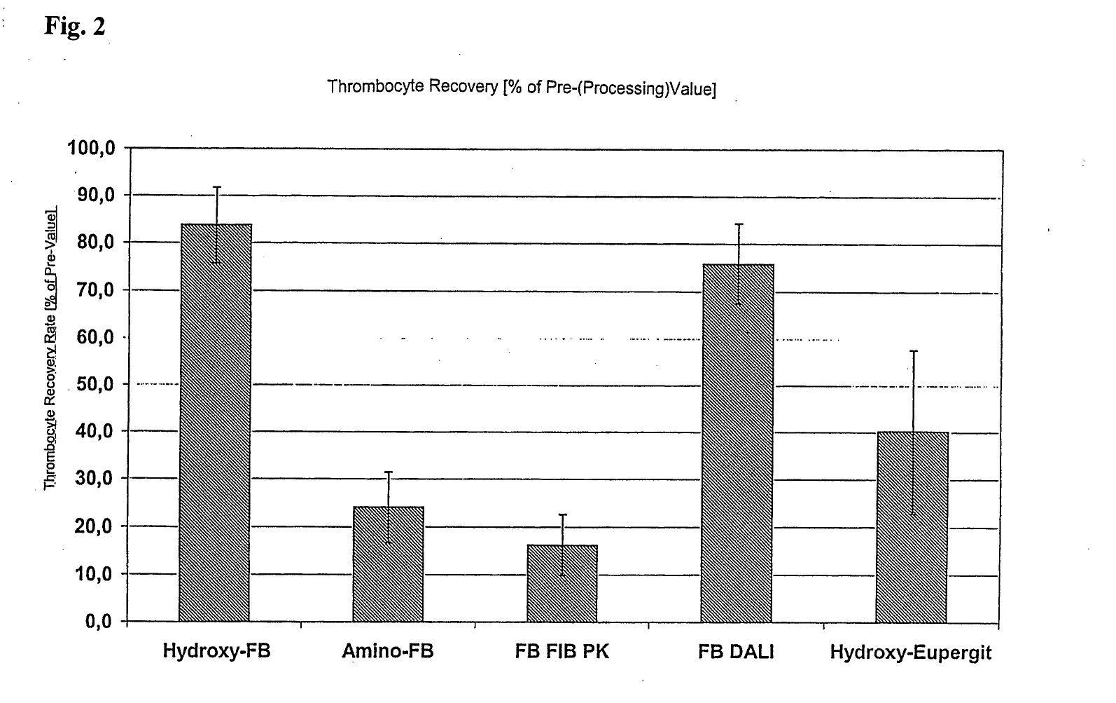

5 ml of heat-sterilized adsorber material (AM) was filled into small chromatography columns. The adsorbent was preprocessed with 80 ml of an electrolyte solution (primer solution) at a flow rate of 5.2 ml/min using a peristaltic pump. This was followed by the processing of 30 ml whole blood (anticoagulated with citrate 15:1) via the column at a flow rate of 3.25 ml/min. Six fractions of 5 ml each were collected. The fibrinogen concentrations in the pre-(processing) values and in the individual fractions were determined using the CLAUSS method (Clauss, A., Rapid Coagulation-Physiological Method for Determining the Fibrinogen: Acta Haematologica (1957) 17, 237-246) on a coagulometer model Thrombotimer 4 (by Behnk-Elektronik, Norderstedt). The bonding capacity is a function of the difference between the pre-values and the averaged post-values and was expressed as a per-gram AM wet weight (WW) of absolutely bound fibrinogen [mg] (ref. FIG. 1). The thrombocyte recovery rate was established by determining the blood count in the pre-value and the individual fractions using a cell analyzer model Sysmex K-1000 (by Sysmex) and was expressed as a percentile reduction from the pre-value (ref. FIG. 2). The test was performed on eight different blood donors. The fibrinogen pre-values determined averaged 328 mg/dl±78 mg/dl.

FIG. 1 shows the lowering of the fibrinogen i.e. the fibrinogen bond in mg, as related to g AM WW, in comparison with certain reference adsorbents.

FIG. 2 shows the thrombocyte recovery rate, as a percentage of the pre-value, in comparison with correspondingly selected reference adsorbents. Legend:
Hydroxy-FB: Adsorbent in the above example according to this invention
Amino-FB: Adsorbent as in Example #2 of EP-A1-1 132 129
FB FIB PK: Adsorbent as in EP-A1-1 132 128 with an organic matrix based on a glycidylmethacrylate-ethyleneglycoldimethacrylate copolymer

FB Dali: Adsorbent as in Example #1 of EP-A1-0 424 698

Eupergit FIB PK: Adsorbent as in EP-A1-1 132 128 with an organic matrix based on Eupergit, marketed by Röhm GmbH & Co. KG of Darmstadt Hydroxy-Eupergit: NaOH hydrolyzate of Eupergit

It is clearly evident from FIG. 1 that the adsorbent of this invention binds fibrinogen nearly as effectively as does the aminated carrier material described in EP-A1-1 132 129 and EP-1 132 128. FIG. 1 also shows that the adsorbent of this invention binds fibrinogen far more strongly than does for instance the Eupergit hydrolyzate sold by Rohm GmbH & Co. KG of Darmstadt. The surprising effect of this invention is manifested in FIG. 2. This novel adsorbent that binds fibrinogen nearly as effectively as the aminated carrier substance described in EP-A1-1 132 129 and EP-A1-1-132 128 outperforms these in terms of a drastically augmented thrombocyte recovery. Thus, the adsorbent according to this invention surprisingly combines a significant reduction of the fibrinogen level with a lower affinity for thrombocytes which, on balance in terms of these performance characteristics, makes it superior to the above reference adsorbents.

Without constituting an absolute statement, one explanation of this surprising effect of the adsorbents according to the present invention may be that the interaction between fibrinogen and thrombocytes works by way of the interaction between GP IIb/IIIA (thrombocytic membrane glycoprotein) and integrin bonding sequences of the D-domain of the fibrinogen molecule. Depending on the bonding strength of fibrinogen on nonphysiologic surfaces, which increases as the degree of hydrophobicity rises, the conformation of the D-domain is apt to change, leading to the point where thrombocytes can no longer interact. As a result, the adsorbents according to this invention combine high fibrinogen bonding with a high thrombocyte recovery rate so that they can also be used for whole-blood applications.

1. An adsorbent for lowering the concentration of fibrinogen and/or fibrin in the blood or blood plasma, encompassing an organic matrix with synthetic side chains covalently bound to the matrix and exhibiting terminal vicinal hydroxy groups formed by the hydrolysis of terminal epoxy groups, said synthetic side chains being free of peptides and devoid of aromatic groups.

2. The adsorbent according to claim 1, in which the organic matrix is a copolymer derived from (meth)acrylic acid esters.

3. The adsorbent according to claim 1, in which the organic matrix has a hydroxyl number in the range from 50 to 1000 μmol/g as related to the dry weight of the adsorber material.

4. The adsorbent according to claim 1, in which the organic matrix is a copolymer derived from at least one epoxy(meth)acrylate and at least one cross-linking agent selected from the group comprising alkylene di(meth)acrylates and polyglycol di(meth)acrylates.

5. The adsorbent according to claim 4, in which the copolymer is a statistical copolymer produced by the polymerization of the monomeric units
(i) glycidylmethacrylate in an amount of 5 to 95 weight percent, preferably 40 to 80 weight percent and most desirably 60 weight percent; and
(ii) ethylene glycoldimethacrylate in an amount of 5 to 95 weight percent, preferably 20 to 60 weight percent and most desirably 40 weight percent,
relative in each case to the total weight of the monomeric units.

6. The adsorbent according to claim 1, in which the organic matrix is composed of spherical, nonaggregated particles.

7. The adsorbent according to claim 1, in which the organic matrix is composed of spherical, nonaggregated particles with a particle size distribution from 50 to 250 μm.

8. The adsorbent according to claim 1, in which the organic matrix is composed of porous, spherical, nonaggregated particles with a particle size distribution from 50 to 250 μm and a pore radius distribution in the range from 10 to 200 nm.

9. Use of the adsorbent according to claim 1 for producing an adsorber serving to lower the concentration of fibrinogen and/or fibrin in the blood or blood plasma.

10. A method for producing the adsorbent according to claim 1, comprising the following steps:
(a) Preparation of the organic matrix with synthetic side chains bound to the matrix and exhibiting terminal epoxy groups;
(b) Hydrolysis of the epoxy groups of the organic matrix while introducing terminal vicinal hydroxy groups into the synthetic side chains covalently bound to the matrix; and, (c) as required, heat treatment of the material obtained in step (b) at a temperature of ≧100° C.

11. The method according to claim 10, whereby the organic matrix prepared in step (a) contains epoxy groups in the amount of 25 to 500 μmol/g as related to the dry weight of the adsorbent material.

12. The method according to claim 10, whereby the organic matrix is a copolymer derived from at least one epoxy(meth)acrylate and at least one cross-linking agent selected from the group comprising alkylene di(meth)acrylates and polyglycol di(meth)acrylates.

13. The method according to claim 12, whereby the copolymer is produced through a suspension polymerization.

14. The method according to claim 12, whereby the copolymer is a random copolymer produced through the polymerization of the monomeric units
(i) glycidyl methacrylate in an amount of 5 to 95% weight percent, preferably 40 to 80 weight percent, and most desirably 60 weight percent, and
(ii) ethylene glycol dimethacrylate in an amount from 5 to 95 weight percent, preferably 20 to 60 weight percent and most desirably 40 weight percent,
relative in each case to the total weight of the monomeric units.

15. The method according to claim 10, whereby the hydrolysis is obtained by incubation, at a temperature in the range from room temperature to 90° C. for a duration ranging from 30 minutes to 24 hours, in 1 to 8 M NaOH and preferably 4 M NaOH.

16. The method according to claim 10, whereby the heat treatment involves sterilization at a temperature in the range from 100 to 140° C.

17. The adsorbent according to claim 2, in which the organic matrix has a hydroxyl number in the range from 50 to 1000 μmol/g as related to the dry weight of the adsorber material.

18. The adsorbent according to claim 17, in which:
the organic matrix is a copolymer derived from at least one epoxy(meth)acrylate and at least one cross-linking agent selected from the group comprising alkylene di(meth)acrylates and polyglycol di(meth)acrylates;
the copolymer is a statistical copolymer produced by the polymerization of the monomeric units
(i) glycidylmethacrylate in an amount of 5 to 95 weight percent, preferably 40 to 80 weight percent and most desirably 60 weight percent; and
(ii) ethylene glycoldimethacrylate in an amount of 5 to 95 weight percent, preferably 20 to 60 weight percent and most desirably 40 weight percent,
relative in each case to the total weight of the monomeric units.

19. The adsorbent according to claim 18, in which:
the organic matrix is composed of spherical, nonaggregated particles;
the organic matrix is composed of spherical, nonaggregated particles with a particle size distribution from 50 to 250 μm;
the organic matrix is composed of porous, spherical, nonaggregated particles with a particle size distribution from 50 to 250 μm and a pore radius distribution in the range from 10 to 200 nm.

20. Use of the adsorbent according to claim 17 for producing an adsorber serving to lower the concentration of fibrinogen and/or fibrin in the blood or blood plasma.

21. Use of the adsorbent according to claim 18 for producing an adsorber serving to lower the concentration of fibrinogen and/or fibrin in the blood or blood plasma.

22. Use of the adsorbent according to claim 19 for producing an adsorber serving to lower the concentration of fibrinogen and/or fibrin in the blood or blood plasma.

23. A method for producing the adsorbent according to claim 18, comprising the following steps:
(a) Preparation of the organic matrix with synthetic side chains bound to the matrix and exhibiting terminal epoxy groups;
(b) Hydrolysis of the epoxy groups of the organic matrix while introducing terminal vicinal hydroxy groups into the synthetic side chains covalently bound to the matrix; and, (c) as required, heat treatment of the material obtained in step (b) at a temperature of ≧100° C.

24. A method for producing the adsorbent according to claim 19, comprising the following steps:
(a) Preparation of the organic matrix with synthetic side chains bound to the matrix and exhibiting terminal epoxy groups;
(b) Hydrolysis of the epoxy groups of the organic matrix while introducing terminal vicinal hydroxy groups into the synthetic side chains covalently bound to the matrix; and,
(c) as required, heat treatment of the material obtained in step (b) at a temperature of ≧100° C.

25. The method according to claim 23, whereby:
the organic matrix prepared in step (a) contains epoxy groups in the amount of 25 to 500 μmol/g as related to the dry weight of the adsorbent material;
the organic matrix is a copolymer derived from at least one epoxy(meth)acrylate and at least one cross-linking agent selected from the group comprising alkylene di(meth)acrylates and polyglycol di(meth)acrylates;
the copolymer is produced through a suspension polymerization;
the copolymer is a random copolymer produced through the polymerization of the monomeric units
(i) glycidyl methacrylate in an amount of 5 to 95% weight percent, preferably 40 to 80 weight percent, and most desirably 60 weight percent, and
(ii) ethylene glycol dimethacrylate in an amount from 5 to 95 weight percent, preferably 20 to 60 weight percent and most desirably 40 weight percent,
relative in each case to the total weight of the monomeric units;
the hydrolysis is obtained by incubation, at a temperature in the range from room temperature to 90° C. for a duration ranging from 30 minutes to 24 hours, in 1 to 8 M NaOH and preferably 4 M NaOH;
the heat treatment involves sterilization at a temperature in the range from 100 to 140° C.

26. The method according to claim 24, whereby:
the organic matrix prepared in step (a) contains epoxy groups in the amount of 25 to 500 μmol/g as related to the dry weight of the adsorbent material;
the organic matrix is a copolymer derived from at least one epoxy(meth)acrylate and at least one cross-linking agent selected from the group comprising alkylene di(meth)acrylates and polyglycol di(meth)acrylates;
the copolymer is produced through a suspension polymerization;
the copolymer is a random copolymer produced through the polymerization of the monomeric units
(i) glycidyl methacrylate in an amount of 5 to 95% weight percent, preferably 40 to 80 weight percent, and most desirably 60 weight percent, and
(ii) ethylene glycol dimethacrylate in an amount from 5 to 95 weight percent, preferably 20 to 60 weight percent and most desirably 40 weight percent,
relative in each case to the total weight of the monomeric units;
the hydrolysis is obtained by incubation, at a temperature in the range from room temperature to 90° C. for a duration ranging from 30 minutes to 24 hours, in 1 to 8 M NaOH and preferably 4 M NaOH;
the heat treatment involves sterilization at a temperature in the range from 100 to 140° C.
